(19) United States
(12) Patent Application Publication
(10) Pub. No.: US 2020/0319201 A1
(43) Pub. Date: Oct. 8, 2020

(54) METHODS FOR MONITORING TUBERCULOSIS MEDICATION

(71) Applicant: INTELLIGENT FINGERPRINTING LIMITED, Cambridge (Cambridgeshire) (GB)

(72) Inventors: Melanie Jane Bailey, Guildford (Surrey) (GB); Mahado Ismail, Guildford (Surrey) (GB); Jeremy Nigel Burgess Walker, Cambridge (Cambridgeshire) (GB)

(21) Appl. No.: 16/955,503

(22) PCT Filed: Dec. 18, 2018
(86) PCT No.: PCT/GB2018/053658

§ 371 Date: Jun. 18, 2020

(30) Foreign Application Priority Data
Dec. 18, 2017 (GB) 1721209.3

(51) Int. Cl. G01N 33/68; G01N 33/569

(52) U.S. Cl. CPC G01N 33/6848; G01N 2800/52; G01N 2333/35; G01N 33/5695

(57) ABSTRACT

The present invention relates to methods of determining whether a subject has taken a dose of Tuberculosis medication through analysis of a sample of sweat obtained from the subject, in the form of a skin-print.

### FIELD OF THE INVENTION

The present invention relates to methods of determining whether a subject has taken a dose of Tuberculosis medication through analysis of a sample of sweat obtained from the subject, in the form of a skin-print.

### BACKGROUND OF THE INVENTION

Tuberculosis (TB) is a disease caused by bacteria called Mycobacterium tuberculosis. The disease usually attacks the lungs, but can also affect other parts of the body. TB is a highly infectious disease, which can spread when a diseased subject coughs, sneezes or talks in the vicinity of others, for example. TB can be treated by taking a course of medication, which can include taking one or more drugs such as isoniazid, pyrazinamide, rifampicin and ethambutol. The course of medication can last for an extended period of time, for example 3, 6 or 9 months, during which the subject may be required to take at least one dose of medication every day. During (or after) the course of medication, if treated effectively, a diseased subject becomes less infectious and therefore less of a danger to others in close proximity. In order for the subject to become less infectious, it is important that the TB medication is taken according to the doctor's orders. If, for example, one or more doses of the medication is not taken at the correct time, the subject can once more become highly infectious and thus endanger those around them.

Compliance with the course of medication is therefore vital if a diseased subject is to be allowed to be around others without posing a risk of further infection.

Current methods to ensure compliance with courses of TB medication include requiring the diseased subject to take each dose of medication in the presence of a third party, e.g. a doctor, nurse or pharmacist. This has the downside that either the diseased subject or the doctor/nurse/pharmacist is required to make regular, potentially inconvenient and time-consuming, trips to see the other. There is therefore a need for a method of compliance which does not require the subject and the doctor/nurse/pharmacist (for example) to travel to be in each other's presence on a regular basis. The nature of some TB patients mean that they often move from one health provider to another and as a result may miss one or more doses of medication and/or not complete their treatment.

It is known that the taking of drugs can be monitored through analysis of bodily fluids such as blood, urine and saliva. However, none of these fluids can be obtained non-invasively by a subject and/or the fluids may represent a biohazard and so may pose difficulties regarding transportation of such biohazardous material. Furthermore, potentially complicated and/or expensive tests (e.g. DNA analysis) may be required to confirm that the fluid has been obtained from the diseased subject, rather than falsified by another party.

It is therefore one object of the present invention to provide a simplified and non-invasive method of ensuring that a diseased subject complies with a course of TB medication.

It is a further object of the present invention to overcome or address the problems of prior art methods of compliance or to at least provide commercially advantageous alternatives thereto.

### SUMMARY OF THE INVENTION

In a first aspect of the invention there is provided a method of determining whether a subject has taken a dose of Tuberculosis medication, the method comprising:
analysing using mass spectrometry a sample of sweat obtained from the subject to determine the presence therein of Tuberculosis medication and/or a metabolite thereof;
wherein the sample of sweat is obtained in the form of a skin-print.

In a second aspect of the invention there is provided a method of determining whether a subject has taken a dose of Tuberculosis medication, the method comprising:
analysing using a lateral flow immunoassay a sample of sweat obtained from the subject to determine the presence therein of Tuberculosis medication and/or a metabolite thereof;
wherein the sample of sweat is obtained in the form of a skin-print.

The present inventors have surprisingly found that mass spectrometry and/or lateral flow immunoassays may be used to ensure compliance with a course of TB medication by analysing a skin-print obtained from the subject. Advantageously, the sweat sample as a skin print, for example a fingerprint or a toe-print, can be obtained from a diseased subject easily and non-invasively, for example, in the subject's own home. A subject's identity is embedded in a skin-print sample, reducing the chances of a falsified test. This skin-print sample can therefore be obtained unsupervised. Furthermore, a skin-print sample is easy to deposit and transport, for example to an analysing laboratory. The sample can then be analysed for ensuring that the subject has taken a dose of TB medication at the necessary time in order to control the Tuberculosis bacteria, therefore reducing the risk of infection to others and also reducing the risk of the bacteria developing resistance to the TB medication.

These factors combine to provide an advantageous method allowing subjects to be monitored for TB medication course compliance from the comfort of their own homes.

Preferred embodiments of the methods according to the invention appear throughout the specification and in particular in the examples.

### Definitions

The term “skin-print” refers to sweat deposited as an impression of a skin's ridge pattern.

Skin-prints include, for example, fingerprints and toe-prints. The term “fingerprint” refers to sweat deposited as an impression of a finger's ridge pattern or a thumb's ridge pattern. The term “toe-print” refers to sweat deposited as an impression of a toe's ridge pattern.

The term “M+H m/z peak” refers to an m/z peak on a mass spectrum caused by an adduct formed by a molecule and a hydrogen ion during mass spectrometry.

The term “taken”, in the context of “a subject has taken a dose of Tuberculosis medication” includes a subject having swallowed, absorbed, injected and/or inhaled a dose of TB medication in accordance with the requirements of the subject's course of medication.

Isoniazid refers to the compound having the formula:

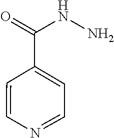

Acetylisoniazid refers to the compound having the formula:

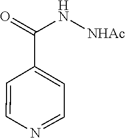 [SMILES: CC(=O)NNC(=O)c1ccncc1]

Pyrazinamide refers to the compound having the formula:

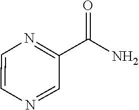

Rifampicin refers to the compound having the formula:

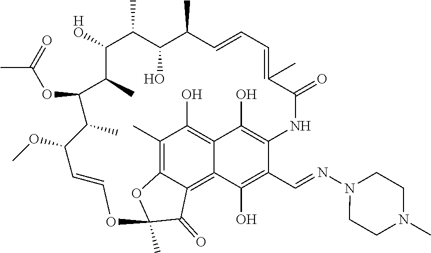 [SMILES: CO[C@H]1/C=C/O[C@@]2(C)Oc3c(C)c(O)c4c(O)c(c(/C=N/N5CCN(C)CC5)c(O)c4c3C2=O)NC(=O)/C(C)=C/C=C/[C@H](C)[C@H](O)[C@@H](C)[C@@H](O)[C@@H](C)[C@H](OC(C)=O)[C@@H]1C]

Ethambutol refers to the compound having the formula:

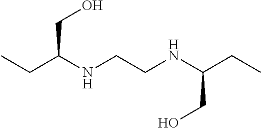

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

The present invention will now be further described. In the following passages different aspects of the invention are defined in more detail. Each aspect so defined may be combined with any other aspect or aspects unless clearly indicated to the contrary. In particular any feature indicated as being preferred or advantageous may be combined with any other feature or features indicated as being preferred or advantageous.

In one embodiment of the present invention, there is provided a method of determining whether a subject has taken a dose of Tuberculosis medication, the method comprising:
analysing using mass spectrometry a sample of sweat obtained from the subject to determine the presence therein of Tuberculosis medication and/or a metabolite thereof;
wherein the sample of sweat is obtained in the form of a skin-print.

It is understood that the sample of sweat is or ‘has been’ obtained in the form of a skin-print, that is, the method is directed to the analysis of a sample of sweat that has previously been provided. Put explicitly, the method is an ex-vivo method. For example, the sample of sweat may have been obtained by the subject contacting a substrate with an area of skin. The substrate/sample may then be provided or transported for analysis and subjected to the method as described herein.

Preferably the subject is a human.

Preferably the skin-print is a fingerprint or a toe-print. More preferably the skin-print is a fingerprint.

Preferably the sample of sweat in the form of a skin-print is obtained without requiring the skin to be washed and cleaned.

In one embodiment, the sample of sweat in the form of a skin-print is obtained after the skin has been washed and cleaned, for example by using soap and water, and before the washed and cleaned skin contacts any other surface. Alternatively, the sample of sweat in the form of a skin-print may be obtained after the skin has been washed and cleaned, for example by using soap and water, then covered by a clean skin protector, such as a glove, for 1 to 20, preferably 5 to 15, more preferably 8 to 12 minutes, and the skin-print then obtained before the washed and cleaned skin contacts any other surface.

Preferably the sample of sweat in the form of a skin-print is obtained on a substrate. Suitable substrates are known in the art. Preferably the substrate comprises or is chromatography paper or plastic, for example chemically inert plastic. More preferably the substrate comprises or is chromatography paper.

Preferably the sample of sweat is obtained from 1 to 48 hours after the subject has (or is meant to have) taken a dose of Tuberculosis medication. More preferably, the sample of sweat is obtained from 1 to 36, or 1 to 24, or 1 to 18, or 1 to 12, or 1 to 8, or 1 to 6, or 1 to 5, or 1 to 4 hours after the subject has (or is meant to have) taken a dose of Tuberculosis medication. Most preferably the sample of sweat is obtained from 1 to 3, or 1.5 to 2.5, or around 2 hours after the subject has (or is meant to have) taken a dose of Tuberculosis medication.

Preferably the Tuberculosis medication comprises isoniazid, pyrazinamide, rifampicin and/or ethambutol. More preferably the Tuberculosis medication comprises isoniazid and/or pyrazinamide.

In a preferred embodiment the Tuberculosis medication comprises isoniazid.

Preferably the dose of Tuberculosis medication comprises at least 300 mg isoniazid.

Preferably the dose of Tuberculosis medication comprises 300 mg to 900 mg isoniazid, more preferably 300 mg to 800 mg isoniazid, more preferably 300 mg to 700 mg isoniazid, more preferably 300 mg to 600 mg isoniazid, more preferably 300 mg to 500 mg isoniazid, most preferably 300 mg to 400 mg isoniazid.

Preferably determining the presence of Tuberculosis medication in the sample of sweat comprises checking for an [M+H]⁺ mass to charge ratio (m/z) peak between 138.06 and 138.08 using mass spectrometry, preferably between 138.06 and 138.07.

In a preferred embodiment the metabolite comprises acetylisoniazid.

Preferably determining the presence of a metabolite of Tuberculosis medication in the sample of sweat comprises checking for an M+H mass to charge ratio (m/z) peak between 180.06 and 180.09 using mass spectrometry, preferably between 180.07 and 180.08.

In a preferred embodiment the Tuberculosis medication comprises pyrazinamide.

Preferably determining the presence of Tuberculosis medication in the sample of sweat comprises checking for an M+H mass to charge ratio (m/z) peak between 124.04 and 124.06 using mass spectrometry, preferably between 124.05 and 124.06.

In a preferred embodiment the metabolite is one or more metabolites of pyrazinamide.

Preferably the sample of sweat has a mass of 0.1 to 2 μg. More preferably the sample of sweat has a mass of 0.2 to 1.8 μg, more preferably 0.4 to 1.6 μg, more preferably 0.5 to 1.5 μg.

Preferably the sample of sweat has a volume of 5 to 50 nl. More preferably the sample of sweat has a volume of 10 to 40 nl, or 15 to 30 nl.

Preferably the sample of sweat comprises 0.1 to 2 ng of the Tuberculosis medication and/or the metabolite thereof. More preferably the sample of sweat comprises 0.2 to 1.8 ng, or 0.4 to 1.6 ng, or 0.6 to 1.4 ng of the Tuberculosis medication and/or the metabolite thereof. Most preferably the sample of sweat comprises 0.8 to 1.2 ng of the Tuberculosis medication and/or the metabolite thereof.

Preferably the mass spectrometry is liquid chromatography mass spectrometry (LC-MS) or paper spray mass spectrometry.

Preferably the mass spectrometry is liquid chromatography mass spectrometry (LC-MS). Analysis using LC-MS is known in the art. LC-MS is known to be quantitative, highly sensitive and selective. LC-MS involves extracting and preparing the sample of sweat prior to analysis by mass spectrometry.

Alternatively, preferably, the mass spectrometry is paper spray mass spectrometry. Paper spray mass spectrometry is described in “Rapid, Secure Drug Testing Using Fingerprint Development and Paper Spray Mass Spectrometry” Catia Costa, Roger Webb, Vladimir Palitsin, Mahado Ismail, Marcel de Puit, Samuel Atkinson, Melanie J. Bailey DOI: 10.1373/clinchem.2017.275578 Published September 2017. Advantageously, paper spray mass spectrometry does not require extraction and sample preparation.

Preferably the method further comprises confirming the identity of the subject by checking the skin-print obtained from the subject. Confirming the identity of the subject may involve photographic development of the skin-print in order to facilitate a photographic or optical comparison with a database.

Preferably the method further comprises analysing the sample of sweat to determine the presence therein of one or more markers which indicate that the level of Tuberculosis infection is declining or increasing. Preferably the one or more markers comprises one or more toxins. Toxins which indicate that the level of Tuberculosis infection is declining or increasing include, for example, MazF6 toxin and tuberculosis necrotizing toxin (TNT) (as described by Sun J1, Siroy A1, Lokareddy RK2, Speer A1, Doornbos KS1, Cingolani G2, Niederweis M1, in “The tuberculosis necrotizing toxin kills macrophages by hydrolyzing NAD”, Nat Struct Mol Biol. 2015 September; 22(9):672-8. doi: 10.1038/nsmb.3064. Epub 2015 Aug. 3.

Preferably the method does not comprise contacting the sample of sweat with one or more microparticles before analysing the sample of sweat using mass spectrometry. More preferably the method does not comprise contacting the sample of sweat with one or more semiconductor microparticles before analysing the sample of sweat using mass spectrometry. More preferably still, the method does not comprise contacting the sample of sweat with one or more porous semiconductor microparticles before analysing the sample of sweat using mass spectrometry.

Preferably the sample of sweat being analysed using mass spectrometry does not comprise and/or is not adhered to one or more one or more microparticles. More preferably the sample of sweat being analysed using mass spectrometry does not comprise and/or is not adhered to one or more one or more semiconductor microparticles. More preferably still the sample of sweat being analysed using mass spectrometry does not comprise and/or is not adhered to one or more one or more porous semiconductor microparticles.

In a further embodiment there is provided a method of determining whether a subject has taken a dose of Tuberculosis medication, the method comprising:
analysing using a lateral flow immunoassay a sample of sweat obtained from the subject to determine the presence therein of Tuberculosis medication and/or a metabolite thereof;
wherein the sample of sweat is obtained in the form of a skin-print.

It is understood that the sample of sweat is or ‘has been’ obtained in the form of a skin-print, that is, the method is directed to the analysis of a sample of sweat that has previously been provided. Put explicitly, the method is an ex-vivo method. For example, the sample of sweat may have been obtained on the lateral flow immunoassay by the subject contacting an area of skin to the lateral flow immunoassay. The lateral flow immunoassay comprising the sample may then be provided or transported for analysis and the sample subjected to the method as described herein.

Suitable lateral flow immunoassays are known in the art. For example, suitable substrates/devices are described in WO2016/012812 and WO2016/135497.

Preferably the subject is a human.

Preferably the skin-print is a fingerprint or a toe-print. More preferably the skin-print is a fingerprint.

Preferably the sample of sweat in the form of a skin-print is obtained after the skin has been washed and cleaned, for example by using soap and water, and before the washed and cleaned skin contacts any other surface. Alternatively, preferably, the sample of sweat in the form of a skin-print is obtained after the skin has been washed and cleaned, for example by using soap and water, then covered by a clean skin protector, such as a glove, for 1 to 20, preferably 5 to 15, more preferably 8 to 12 minutes, and the skin-print then obtained before the washed and cleaned skin contacts any other surface.

Preferably the sample of sweat is obtained from 1 to 48 hours after the subject has (or is meant to have) taken a dose of Tuberculosis medication. More preferably, the sample of sweat is obtained from 1 to 36, or 1 to 24, or 1 to 18, or 1 to 12, or 1 to 8, or 1 to 6, or 1 to 5, or 1 to 4 hours after the subject has (or is meant to have) taken a dose of Tuberculosis medication. Most preferably the sample of sweat is obtained from 1 to 3, or 1.5 to 2.5, or around 2 hours after the subject has (or is meant to have) taken a dose of Tuberculosis medication.

Preferably the Tuberculosis medication comprises isoniazid, pyrazinamide, rifampicin and/or ethambutol. More preferably the Tuberculosis medication comprises isoniazid and/or pyrazinamide.

In a preferred embodiment the Tuberculosis medication comprises isoniazid.

Preferably the dose of Tuberculosis medication comprises at least 300 mg isoniazid.

Preferably the dose of Tuberculosis medication comprises 300 mg to 900 mg isoniazid, more preferably 300 mg to 800 mg isoniazid, more preferably 300 mg to 700 mg isoniazid, more preferably 300 mg to 600 mg isoniazid, more preferably 300 mg to 500 mg isoniazid, most preferably 300 mg to 400 mg isoniazid.

In a preferred embodiment the metabolite comprises acetylisoniazid.

In a preferred embodiment the Tuberculosis medication comprises pyrazinamide.

In a preferred embodiment the metabolite is one or more metabolites of pyrazinamide.

Preferably the sample of sweat has a mass of 0.1 to 2 μg. More preferably the sample of sweat has a mass of 0.2 to 1.8 μg, more preferably 0.4 to 1.6 μg, more preferably 0.5 to 1.5 μg.

Preferably the sample of sweat has a volume of 5 to 50 nl. More preferably the sample of sweat has a volume of 10 to 40 nl, or 15 to 30 nl.

Preferably the sample of sweat comprises 0.1 to 2 ng of the Tuberculosis medication and/or the metabolite thereof. More preferably the sample of sweat comprises 0.2 to 1.8 ng, or 0.4 to 1.6 ng, or 0.6 to 1.4 ng of the Tuberculosis medication and/or the metabolite thereof. Most preferably the sample of sweat comprises 0.8 to 1.2 ng of the Tuberculosis medication and/or the metabolite thereof.

Preferably the method further comprises confirming the identity of the subject by checking the skin-print obtained from the subject.

Preferably the method further comprises analysing the sample of sweat to determine the presence therein of one or more markers which indicate that the level of Tuberculosis infection is declining or increasing. Preferably the one or more markers comprises one or more toxins.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents.

These and other aspects of the invention will now be described with reference to the accompanying Figures, in which:

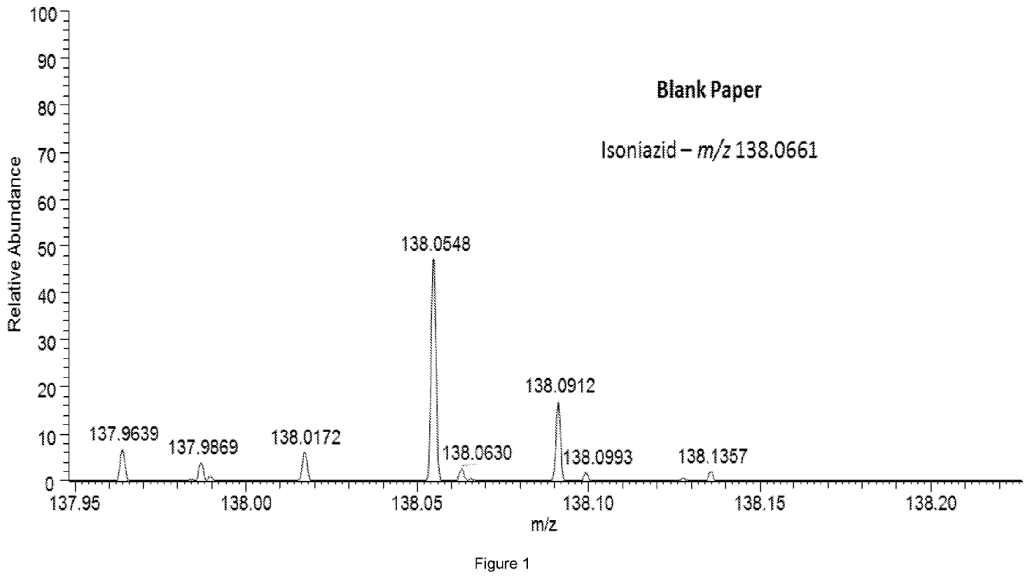

FIG. 1: is a mass spectrum resulting from preparation of a blank piece of chromatography paper, focussed on the range where Isoniazid (if present) is observed.

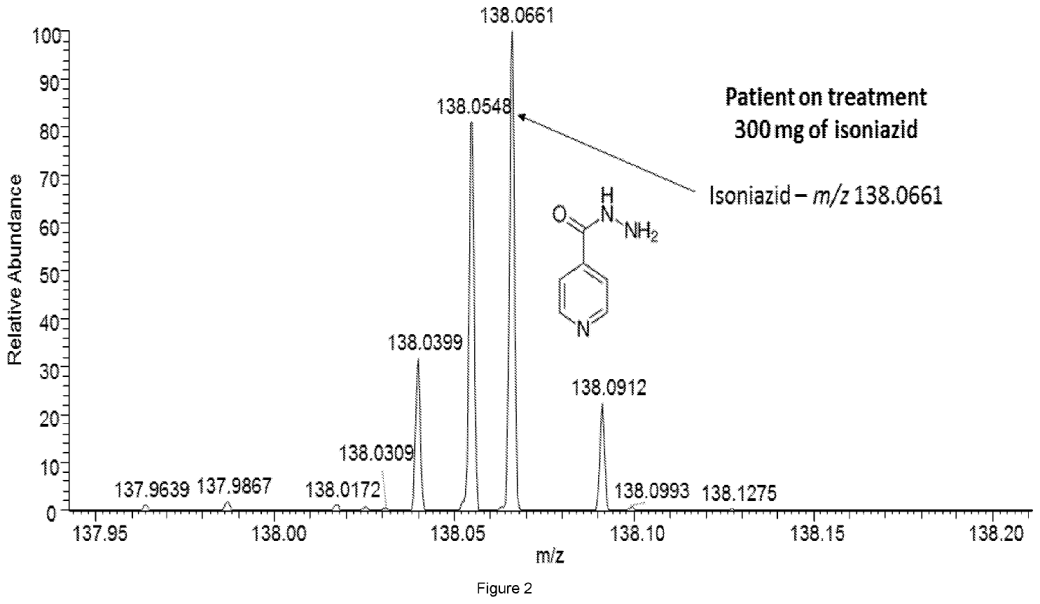

FIG. 2: is a mass spectrum resulting from preparation of a piece of chromatography paper having a sample of sweat thereon, in the form of a fingerprint. The sample was obtained from a subject (TB2017-FP36) who had taken 300 mg of Isoniazid approximately 3 hours beforehand. The spectrum is focussed on the range where Isoniazid (if present) is observed.

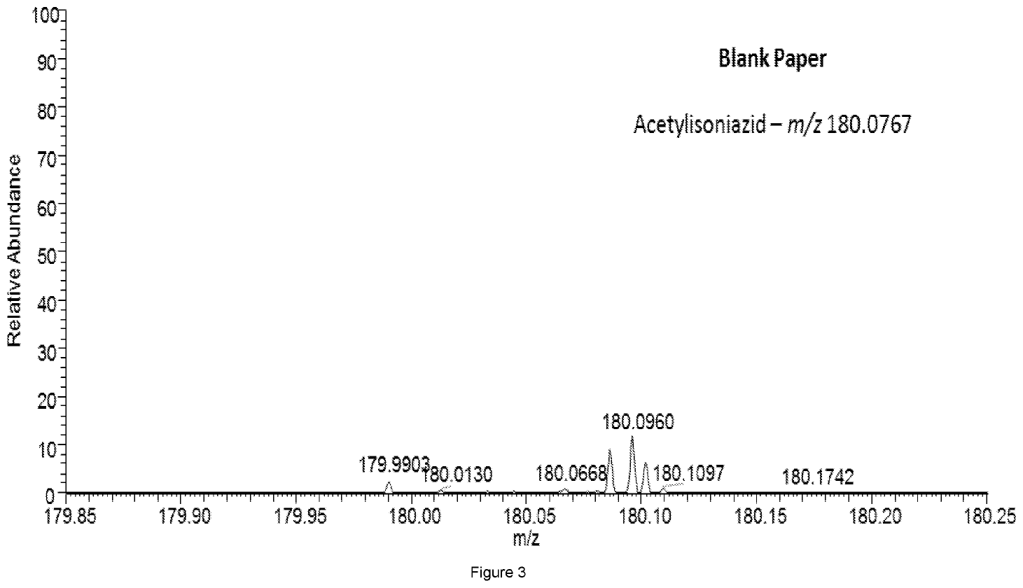

FIG. 3: is a mass spectrum resulting from preparation of a blank piece of chromatography paper, focussed on the range where Acetylisoniazid (if present) is observed.

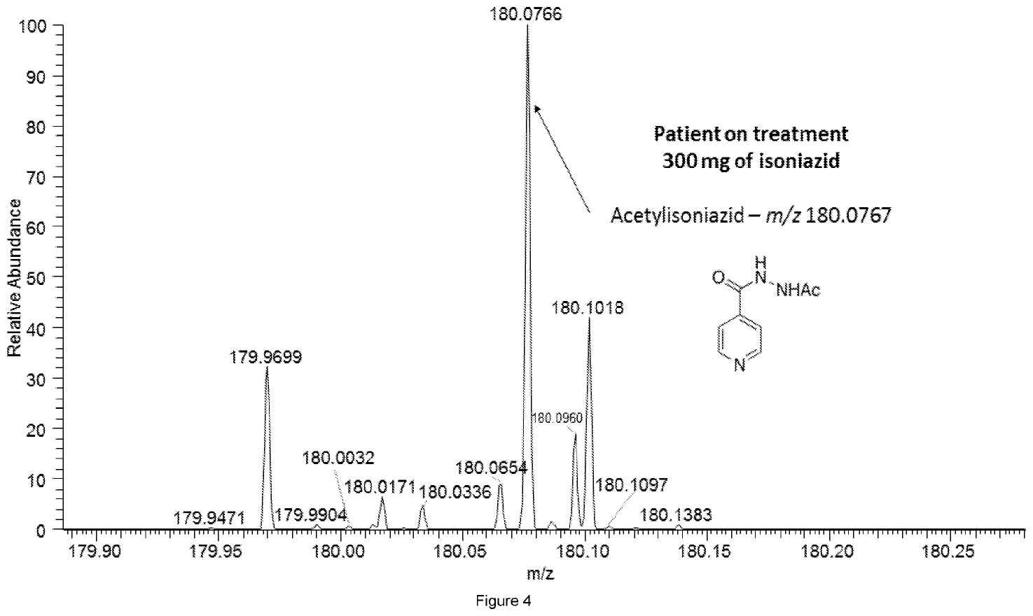

FIG. 4: is a mass spectrum resulting from preparation of a piece of chromatography paper having a sample of sweat thereon, in the form of a fingerprint. The sample was obtained from a subject (TB2017-FP36) who had taken 300 mg of Isoniazid approximately 3 hours beforehand. The spectrum is focussed on the range where Acetylisoniazid (if present) is observed.

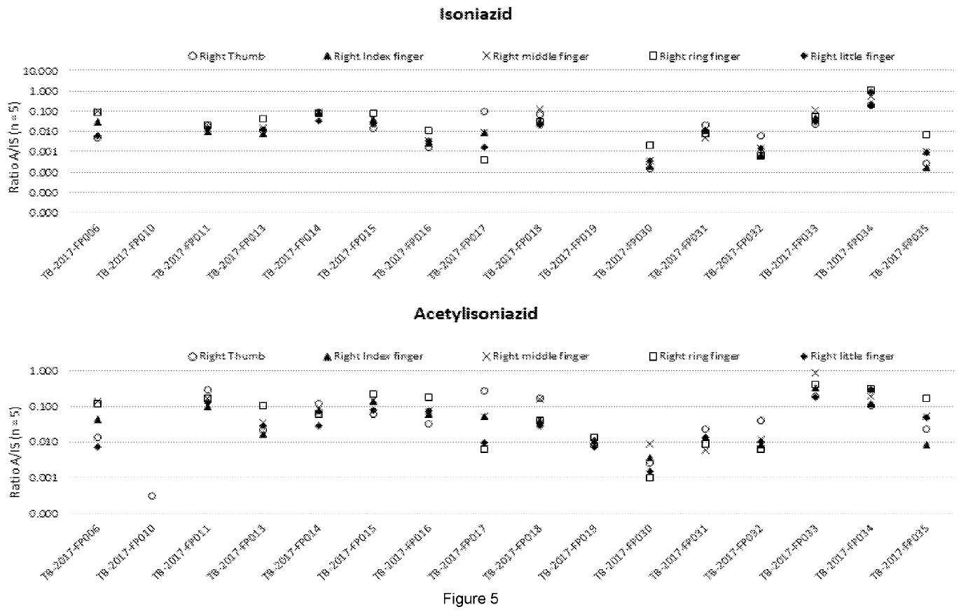

FIG. 5: is a diagram showing relative amounts of Isoniazid and Acetylisoniazid present in fingerprint samples obtained from 16 subjects. The fingerprint samples were obtained without the subjects washing their hands.

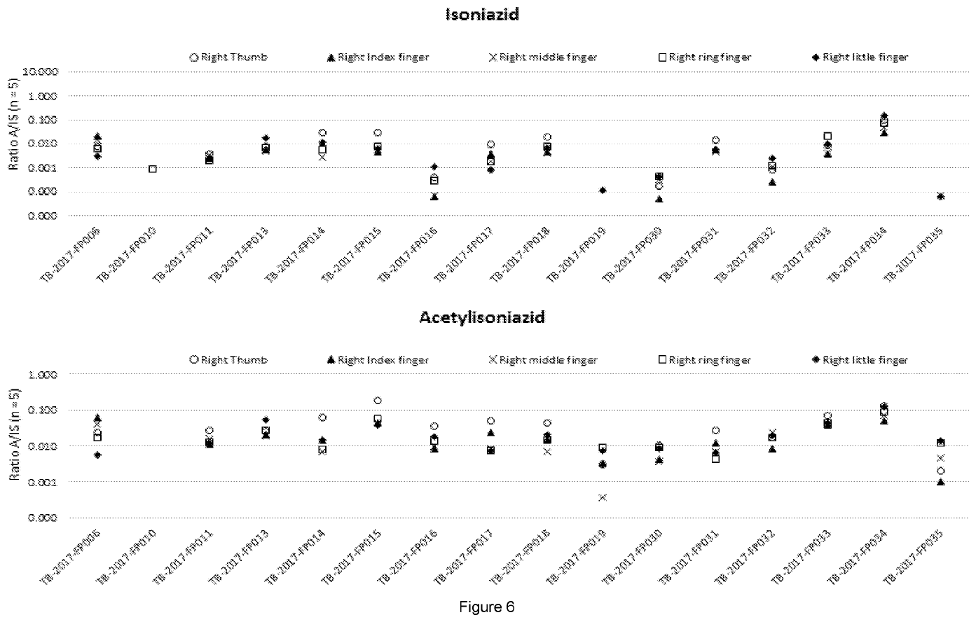

FIG. 6: is a diagram showing relative amounts of Isoniazid and Acetylisoniazid present in fingerprint samples obtained from 16 subjects. The fingerprint samples were obtained after the subjects had washed their hands using soap and water.

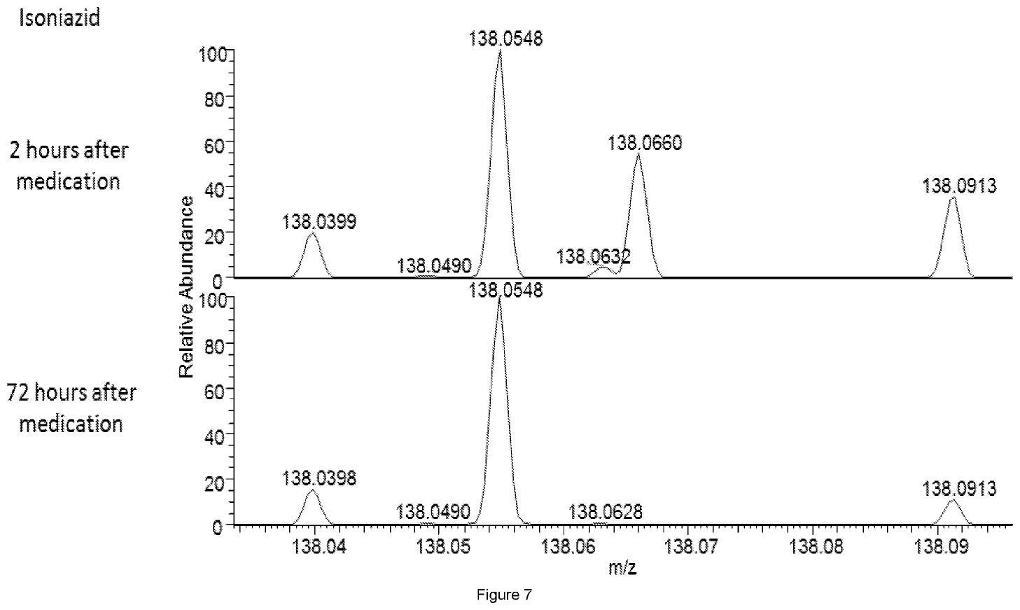

FIG. 7: shows two mass spectra for two samples of fingerprint sweat, focussed on the range where Isoniazid (if present) is observed. The first sample was obtained from a subject who had taken Isoniazid 2 hours previously; the second sample was obtained from a subject who had taken Isoniazid 72 hours previously.

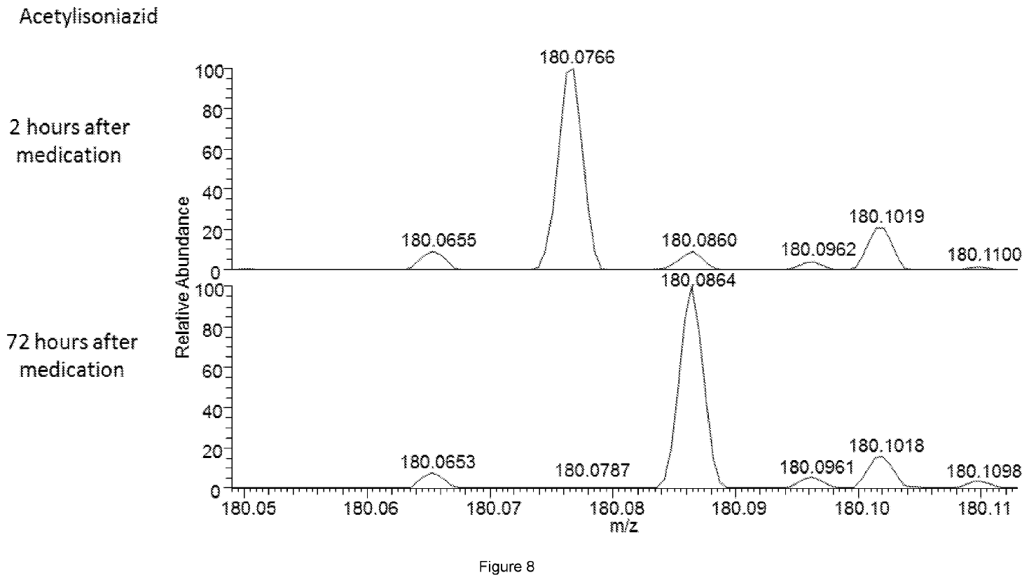

FIG. 8: shows two mass spectra for two samples of fingerprint sweat, focussed on the range where Acetylisoniazid (if present) is observed. The first sample was obtained from a subject who had taken Isoniazid 2 hours previously; the second sample was obtained from a subject who had taken Isoniazid 72 hours previously.

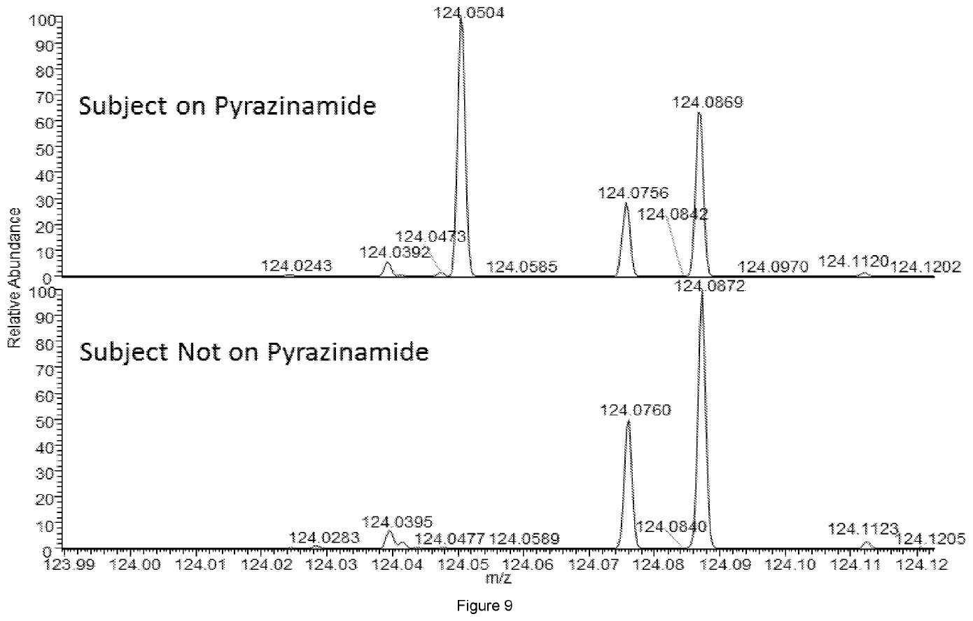

FIG. 9: shows two mass spectra for two samples of fingerprint sweat. The first sample was obtained from a subject who had taken pyrazinamide; the second sample was obtained from a subject who had not taken pyrazinamide.

The following non-limiting examples further illustrate the present invention.

### EXAMPLES

Experimental Methods

Fingerprint Collection Method

All subjects from whom fingerprint samples were taken were human. Fingerprints were taken from the five fingers (i.e. the four fingers and the thumb) of each subject's right hand:
without the hand having been washed; or
after the hand has been washed with soap and water, and covered with a clean surgical glove for 10 minutes, without contacting any other surface.

Liquid Chromatography Mass Spectrometry Analysis of Samples

The chromatography paper with fingerprint samples thereon were placed in a 2 ml Eppendorf microcentrifuge tube, following which an extraction solution (1.5 ml of 10% dichloromethane in methanol) was added. The tube was then centrifuged for 2 min (at 9.5 centrifugal force). The solvent extract was evaporated to dryness under a stream of nitrogen at room temperature (20° C.) and reconstituted in 100 μl mobile phase solution (95:5 water/acetonitrile+0.1% formic acid+iosotopically labelled internal standards) before being vortexed and transferred to a 300 μl glass micro-insert vial, with 5 μl being injected onto an LC-MS/MS system.

Chromatographic separation was performed on a Thermo Scientific™ Ultimate3000 UHPLC system equipped with a binary solvent manager, column manager and autosampler. The injection volume was 5 μL. Separation was performed on a Kinetex XB-C₁₈ column (100×2.1 mm, 5 μm) operated at 30° C. at a flow rate of 0.25 ml/min. Mobile phase comprises 95% H₂O (0.1% formic acid) and 5% acetonitrile (ACN) (0.1% formic acid).

The prepared samples were introduced to a Thermo Orbitrap Q-Exactive Plus mass spectrometer using the standard ESI interface with a capillary temperature of 320° C. and spray voltage 3 kV. Positive mass spectra were acquired in full scan mode within a range of m/z 50-500 at a mass resolution of 70 000 at m/z 200.

### Example 1

Fingerprint samples were obtained from 15 subjects who had taken 300 mg isoniazid approximately 3 hours beforehand and one subject (TB2017-FP019) who had taken 400 mg isoniazid, without their hands having been washed. Of the 15 subjects who had taken 300 mg isoniazid, one (TB2017-FP010) was resistant to isoniazid. TB2017-FP010's results were therefore discounted from the analysis.

This provided 75 fingerprint samples (one for each finger/thumb of the subjects' right hands). Each of the 75 fingerprint samples was analysed using liquid chromatography mass spectrometry, checking for the presence of Isoniazid and Acetylisoniazid (a metabolite of Isoniazid).

The presence of Isoniazid is evidenced by a peak at an m/z ratio of 138.0661, as shown in FIG. 2, which is the mass spectrum resulting from the analysis of a fingerprint sample from subject TB2017-FP36. The presence of Acetylisoniazid is evidenced by a peak at an m/z ratio of 180.0766, as shown in FIG. 4, which is the mass spectrum resulting from the analysis of a fingerprint sample from subject TB2017-FP36.

The results from analysis of the 75 samples are displayed in FIG. 5. The analysis found that Isoniazid was detected in 51 out of the 75 fingerprint samples (68%) and Acetylisoniazid was detected in 73 out of the 75 fingerprint samples (97%).

### Example 2

Fingerprint samples were obtained from the same 16 subjects as Example 1, after they had washed their hands using soap and water. Again, TB2017-FP010's results were discounted from the analysis. This provided 75 fingerprint samples (one for each finger/thumb of the subjects' right hands). Each of the 75 fingerprint samples was analysed using liquid chromatography mass spectrometry, checking for the presence of Isoniazid and Acetylisoniazid. The results are displayed in FIG. 6. The analysis found that Isoniazid was detected in 40 out of the 75 fingerprint samples (53%) and Acetylisoniazid was detected in 72 out of the 75 fingerprint samples (96%).

### Example 3

Fingerprint samples were obtained from 5 subjects who had finished a course of Isoniazid at least 72 hours previously.

25 fingerprints were obtained from the subjects, without the subjects having washed their hands. These samples were analysed using mass spectrometry. Isoniazid was detected in 0 fingerprint samples. Acetylisoniazid was detected in 0 fingerprint samples.

The 5 subjects then washed their hands using soap and water. 25 fingerprints were then obtained from the subjects. These samples were analysed using mass spectrometry. Isoniazid was detected in 0 fingerprint samples. Acetylisoniazid was detected in 0 fingerprint samples.

### Example 4

Fingerprint samples were obtained from 3 subjects who had never taken Isoniazid. 15 fingerprints were obtained from the subjects, without the subjects having washed their hands. These samples were analysed using mass spectrometry. Isoniazid was detected in 0 fingerprint samples. Acetylisoniazid was detected in 0 fingerprint samples.

The 3 subjects then washed their hands using soap and water. 15 fingerprints were then obtained from the subjects. These samples were analysed using mass spectrometry. Isoniazid was detected in 0 fingerprint samples. Acetylisoniazid was detected in 0 fingerprint samples.

### Example 5

Two samples of fingerprint sweat were obtained from the same subject. The first sample was obtained 2 hours after the subject had taken Isoniazid. The second sample was obtained from the subject 72 hours after the subject had taken Isoniazid. The two samples were analysed using liquid chromatography mass spectrometry and the results analysed. The results are shown in FIG. 7. The presence of Isoniazid is evidenced by a peak at an m/z ratio of 138.0660. Analysis of the first sample, obtained 2 hours after the subject had taken Isoniazid, shows the presence of Isoniazid. In contrast, analysis of the second sample, obtained 72 hours after the subject had taken Isoniazid, does not show the presence of Isoniazid.

### Example 6

Two samples of fingerprint sweat were obtained from the same subject. The first sample was obtained 2 hours after the subject had taken Isoniazid. The second sample was obtained from the subject 72 hours after the subject had taken Isoniazid. The two samples were analysed using liquid chromatography mass spectrometry and the results analysed.

The results are shown in FIG. 8. The presence of Acetylisoniazid is evidenced by a peak at an m/z ratio of 180.0766. Analysis of the first sample, obtained 2 hours after the subject had taken Isoniazid, shows the presence of Acetylisoniazid. In contrast, analysis of the second sample, obtained 72 hours after the subject had taken Isoniazid, does not show the presence of Acetylisoniazid.

### Example 7

A fingerprint sample was obtained from a subject who had taken 1,000 mg pyrazinamide approximately 3 hours beforehand. The sample was taken after the subject had washed their hands. The fingerprint sample was analysed using liquid chromatography mass spectrometry, checking for the presence of pyrazinamide. The mass spectrometry analysis of the fingerprint sample is shown in FIG. 9. The presence of pyrazinamide is evidenced by a peak at an m/z ratio of 124.0505. FIG. 9 also shows a second mass spectrum which shows the mass spectrometry analysis of a sample obtained from a subject who had not taken pyrazinamide.

1. A method of determining whether a subject has taken a dose of Tuberculosis medication, the method comprising:
analysing using mass spectrometry a sample of sweat obtained from the subject to determine the presence therein of Tuberculosis medication and/or a metabolite thereof;
wherein the sample of sweat is obtained in the form of a skin-print.

2. The method of claim 1, wherein the skin-print is a fingerprint.

3. The method of any of the preceding claims, wherein the Tuberculosis medication comprises isoniazid, pyrazinamide, rifampicin and/or ethambutol.

4. The method of any of the preceding claims, wherein the Tuberculosis medication comprises isoniazid and/or pyrazinamide.

5. The method of any of the preceding claims, wherein the Tuberculosis medication comprises isoniazid.

6. The method of any of the preceding claims, wherein the dose of Tuberculosis medication comprises at least 300 mg isoniazid.

7. The method of any of the preceding claims, wherein determining the presence of Tuberculosis medication in the sample of sweat comprises checking for an M+H mass to charge ratio (m/z) peak between 138.06 and 138.08 using mass spectrometry, preferably between 138.06 and 138.07.

8. The method of any of the preceding claims, wherein the metabolite comprises acetylisoniazid.

9. The method of any of the preceding claims, wherein determining the presence of a metabolite of Tuberculosis medication in the sample of sweat comprises checking for an M+H mass to charge ratio (m/z) peak between 180.06 and 180.09 using mass spectrometry, preferably between 180.07 and 180.08.

10. The method of any of the preceding claims, wherein the Tuberculosis medication comprises pyrazinamide.

11. The method of any of the preceding claims, wherein determining the presence of Tuberculosis medication in the sample of sweat comprises checking for an M+H mass to charge ratio (m/z) peak between 124.04 and 124.06 using mass spectrometry, preferably between 124.05 and 124.06.

12. The method of any of the preceding claims, wherein the sample of sweat has a mass of 0.1 to 2 μg.

13. The method of any of the preceding claims, wherein the sample of sweat has a volume of 5 to 50 nl.

14. The method of any of the preceding claims, wherein the sample of sweat comprises 0.1 to 2 ng of the Tuberculosis medication and/or the metabolite thereof.

15. The method of any of the preceding claims, wherein the mass spectrometry is liquid chromatography mass spectrometry.

16. The method of any of the preceding claims, wherein the mass spectrometry is paper spray mass spectrometry.

17. The method of any of the preceding claims, further comprising confirming the identity of the subject by checking the skin-print obtained from the subject.

18. The method of any of the preceding claims, further comprising analysing the sample of sweat to determine the presence therein of one or more markers which indicate that the level of Tuberculosis infection is declining or increasing.

19. The method of claim 18, wherein the one or more markers comprises one or more toxins.

20. A method of determining whether a subject has taken a dose of Tuberculosis medication, the method comprising:
analysing using a lateral flow immunoassay a sample of sweat obtained from the subject to determine the presence therein of Tuberculosis medication and/or a metabolite thereof;
wherein the sample of sweat is obtained in the form of a skin-print.

21. The method of claim 20, wherein the skin-print is a fingerprint.

22. The method of claim 20 or claim 21, further comprising confirming the identity of the subject by checking the skin-print obtained from the subject.

23. The method of any of claims 20 to 22, further comprising analysing the sample of sweat to determine the presence therein of one or more markers which indicate that the level of Tuberculosis infection is declining or increasing.

24. The method of claim 23, wherein the one or more markers comprises one or more toxins.
